(19) United States
(12) Patent Application Publication
(10) Pub. No.: US 2026/0134285 A1
(43) Pub. Date: May 14, 2026

(54) METHOD FOR EXTRACTING INFORMATION FROM MULTI-CHANNEL MEASUREMENT DATA, MEASUREMENT SYSTEM FOR OBTAINING MULTI-CHANNEL MEASUREMENT DATA

(71) Applicant: Oxford University Innovation Limited, Oxford (GB)

(72) Inventors: Lei LU, Oxford (Oxfordshire) (GB); Tingting ZHU, Oxford (Oxfordshire) (GB); David CLIFTON, Oxford (Oxfordshire) (GB); Antônio Horta RIBEIRO, Uppsala (SE); Antonio Luiz Pinho RIBEIRO, Belo Horizonte (BR); Yuan-Ting ZHANG, Kowloon (Hong Kong) (CN)

(21) Appl. No.: 19/121,157

(22) PCT Filed: Sep. 7, 2023
(86) PCT No.: PCT/GB2023/052309

§ 371 Date: Apr. 15, 2025

(30) Foreign Application Priority Data
Oct. 26, 2022 (GB) 2215839.8

(51) Int. Cl. G06N 3/084; G06N 3/0442; G06N 3/0464; G06N 3/048; G16H 50/20

(52) U.S. Cl. CPC G06N 3/084; G06N 3/0442; G06N 3/0464; G06N 3/048; G16H 50/20

(57) ABSTRACT

Methods and apparatus for extracting information from multi-channel measurement data are disclosed. In one arrangement, the measurement data is input to a set of trained first machine learning models, each channel to a different first machine learning model. Outputs provide respective sets of extracted feature maps, one set for each of the channels and each set comprising one extracted feature map for each of a plurality of convolution kernels. The outputs are combined and input to a trained second machine learning model, which provides a model prediction about the state of a subject. Interpretation information is generated that indicates relative strengths of contribution to the model prediction from different portions of the measurement data in each channel. The interpretation information for each channel is generated based on a weighted sum of the extracted feature maps output from the respective first machine learning model.

The present disclosure relates to methods and apparatus for obtaining and analysing multi-channel measurement data, such as measurement data obtained from electrocardiogram (ECG), electroencephalography (EEG) and/or photoplethysmography (PPG) measurements.

AI-driven approaches, and in particular deep learning, are developing at pace, have increasing potential for application in healthcare, and have been used to address data analysis challenges relevant for a variety of medical conditions. Despite the great promise of AI techniques in healthcare, concerns over the unknown interpretation process, i.e., black-box model, have spurred a movement toward building trust in machine learning (ML) algorithms. There are growing calls for transparent and trustworthy AI models from clinicians, lawmakers, and government regulators. Transparency can support a physician's competence in interpretation, and build trust within the physician-patient relationship; conversely, a lack of this interpretive ability may impede the general acceptance of AI techniques in healthcare practice. In addition, the improvement in interpretability of clinical data allows physicians to better understand the biological mechanisms behind disease, to identify disease-specific features, and enables efforts with the potential to derive more reliable biomarkers.

Various approaches have been taken to develop interpretable techniques that produce explanations for ML decisions. Examples include the following.
1) Class activation mapping (CAM), as described in Bolei Zhou et al. “Learning deep features for discriminative localization”. In: Proceedings of IEEE Conference on Computer Vision and Pattern Recognition. 2016, pp. 2921-2929.
2) Local interpretable model-agnostic explanation (LIME), as described in Marco Tulio Ribeiro, Sameer Singh, and Carlos Guestrin. ““Why should I trust you?” Explaining the predictions of any classifier”. In: Proceedings of the 22nd ACM SIGKDD International Conference on Knowledge Discovery and Data Mining. 2016, pp. 1135-1144.
3) Shapley additive explanations (SHAP), as described in Scott M Lundberg and Su-In Lee. “A unified approach to interpreting model predictions”. Advances in Neural Information Processing Systems 30 (2017).
4) Gradient-weighted class activation mapping (Grad-CAM), as described in Ramprasaath R Selvaraju et al. “Grad-CAM: Visual explanations from deep networks via gradient-based localization”. Proceedings of IEEE International Conference on Computer Vision (2017), pp. 618-626.

Grad-CAM and its variants have shown promising interpretation ability in processing medical images. For example, it has been used to localise salient areas in chest radiographs for acute respiratory distress syndrome (ARDS) diagnosis, to segment chest X-ray images for COVID-19 detection, and to identify scaphoid fractures in radiographic images. However, these studies either focused on specific tasks or have limited experimental validation, and the interpretation capabilities of these techniques are still largely unexplored.

ECG recording is the most commonly performed diagnostic test to screen cardiovascular diseases (CVD), which are responsible for more than 30% of all deaths globally. It is understood that ECG recording provides an assessment of overall rhythm and cardiovascular status; nevertheless, interpretation of the test varies greatly, even among cardiology specialists. Such variance between physicians presents a challenge to ensure consistency and reliability in the diagnosis. Moreover, the physician's recognition of abnormal morphologies is mostly limited to existing cardiac disorders; it is therefore difficult to detect rare or relatively unknown diseases or recognise visually imperceptible elements in the morphology. At the same time, modern technologies are constantly increasing the ability to acquire large numbers of ECG recordings, with more than 300 million ECGs being obtained annually worldwide.

Recent studies have shown advances in using AI techniques for digital ECG analysis, applied for example for abnormal heart rhythm detection, cardiac contractile dysfunction identification, aortic valve stenosis screening, and early diagnosis of low ejection fraction. However, most of these AI models focus on task performance rather than extracting clinically useful information or expanding knowledge from ECG recordings. For example, the AI models may demonstrate cardiologist-level detection of abnormalities using ECGs but output diagnostic scores instead of explaining how the ECG morphologies were used for the diagnosis. Despite the impressive performance of AI models, it is unreasonable for either a patient or medical professional to accept an automated diagnosis at face value without justification. More importantly, AI techniques are often highly complex, and thus require a substantial number of samples to train the model, without which outputs may be unreliable and have potential pitfalls. For example, a treatment recommendation with an explicit contraindication could be made even by well-trained AI systems, but without an accompanying means of alerting the treating physician of the potential risk there may be a consequence of major harm to patients.

It is an object of the present disclosure to at least partially address one or more of the challenges described above.

According to an aspect of the invention, there is provided a computer-implemented method for extracting information from multi-channel measurement data, the method comprising: receiving multi-channel measurement data derived from measurements performed on a subject; providing the measurement data as input to a set of trained first machine learning models, each channel of the measurement data being provided exclusively to a different respective one of the first machine learning models, and obtaining as outputs from the first machine learning models respective sets of extracted feature maps, one set for each of the channels and each set comprising one extracted feature map for each of a plurality of convolution kernels; combining the outputs from the first machine learning models to obtain a combined output, inputting the combined output to a trained second machine learning model, and obtaining from the second machine learning model a model prediction about the state of the subject; and generating and outputting interpretation information indicating relative strengths of contribution to the model prediction from different portions of the measurement data in each channel, the interpretation information for each channel being generated based on a weighted sum of the extracted feature maps output from the respective first machine learning model.

Thus, a method is provided in which features are extracted independently from different channels of the measurement data in an isolation stage (using the first machine learning models) and a model prediction is obtained by applying these extracted features to a trained machine learning model that takes account of interrelationships between the different channels (the second machine learning model). Separating the different channels of measurement data and providing them as independent inputs to the different first machine learning models allows features to be learned more precisely from the different channels, as well as avoiding the need to share kernel weights across different channels, which can reduce the ability to interpret each channel precisely. At the same time, the second machine learning model provides the ability to explore elaborate relationships between different channels and thereby provide an accurate model prediction. The architecture is demonstrated to achieve an advantageous balance of performance in terms of the quality of model prediction and the provision of effective interpretation information on a channel-by-channel basis.

In an embodiment, the generating and outputting of the interpretation information comprises dimensionally aligning weighted activation maps to the measurement data, each weighted activation map representing the weighted sum of the extracted feature maps output from a respective one of the first machine learning models. The dimensional alignment may be performed along a time dimension for example. Dimensionally aligning the weighted activation maps to the measurement data makes it easier to visualise relationships between the weighted activation maps and the measurement data (i.e., how they map to each other). For example, in the context of interpreting ECG data, the dimensional alignment makes it easier to recognise which parts of an ECG trace are relevant for a diagnosis corresponding to the model prediction (e.g., an abnormality or hypertension).

In an embodiment, the first machine learning models are configured such that at least one dimension of the extracted feature maps is at least 0.2 times a corresponding dimension of the measurement data. Minimizing a difference in size between the extracted feature maps and the measurement data minimizes a difference in resolution between the extracted feature maps and the measurement data, thus providing higher quality interpretation information.

In an embodiment, each first machine learning model comprises only one pooling layer or only two pooling layers. This is an unusual approach in the sense that deep learning models usually use larger numbers of pooling layers to reduce the dimension of input data and efficiently achieve high quality predictions. However, having larger numbers of pooling layers results in the extracted feature maps calculated from the last Conv layer having a much smaller size than the input data, which then requires a correspondingly larger linear rescaling to achieve dimensional alignment. For example, the VGG model is widely used for image processing; it has a size of 224×224 for the input image, and the dimension of kernels in the last Conv layer is 14×14, which is one sixteenth of the size of the input image. Therefore, the VGG model needs to magnify the heatmap at sixteen times for each dimension, which indicates a large number of adjacent data points sharing the same value of heatmap, and thus reduces the resolution for the interpretation. By using fewer pooling layers, the present embodiment provides a higher quality visualisation of salient features (with higher resolution).

In an embodiment, the multi-channel measurement data comprises data derived from simultaneous performance of measurements having different measurement types, each measurement type corresponding to a different respective one of the channels. The different measurement types may comprise measurement obtained from multiple ECG channels, each ECG channel corresponding to a different respective one of the measurement types. The model prediction may comprise information about ECG abnormality and/or hypertension. The inventors have found that the method works particularly effectively for identifying ECG abnormalities and for identifying hypertension, as well as being able to provide interpretation information that indicates effectively which aspects of the ECG data are most relevant for the identified abnormalities and/or hypertension. The inventors have demonstrated generation of interpretation information indicating salient features that match well with existing knowledge as well as salient features that may provide new clinical implications. For example, the diagnosis of SB and ST primarily focuses on the checking of a patient's heart rate by cardiologists, whereas methods of the present disclosure highlight the importance of U waves for the identification of SB.

From a clinical perspective, the interpretation of ECG recordings is critical to understand and diagnose cardiovascular diseases. Application of the method to ECG data provides for augmenting the current clinical workflow in several ways. First, rather than developing a stand-alone computerised method for automated ECG diagnosis, methods of the present disclosure enable a more holistic approach by producing visually salient features that support interpretation of ECG recordings, which allows practitioners to understand the decision that has been made by the AI model, and therefore reduce the risk of misdiagnosis. Second, benefiting from the identification of salient features and the identified dominant ECG leads, the method has the potential to facilitate the discovery of new biomarkers, particularly in areas where expert knowledge is not readily available, i.e., hypertension screening using ECG recordings. Third, even in well-established areas, e.g., diagnosis of arrhythmias, the methods of the present disclosure are shown to provide new insights for the interpretation of ECG morphologies; this enables promotion of further understanding of cardiovascular systems. Notably, the model disclosed in the present document does not involve any prior domain knowledge, i.e., cardiovascular medicine, but instead allows automated learning of salient features in data measurements that are collected from physically isolated sensors. The approach can therefore be applied in other scenarios apart from the medical tasks demonstrated in this study, for example in EEG and PPG or other scenarios involving multiple isolated sensors or measurement channels.

According to an alternative aspect of the invention, there is provided a measurement system for obtaining multi-channel measurement data, comprising: a measurement apparatus configured to perform multi-channel measurements on a subject to obtain multi-channel measurement data; and a data processing unit comprising a processor configured to perform the following steps: provide the measurement data as input to a set of trained first machine learning models, each channel of the measurement data being provided exclusively to a different respective one of the first machine learning models, and obtain as outputs from the first machine learning models respective sets of extracted feature maps, one set for each of the channels and each set comprising one extracted feature map for each of a plurality of convolution kernels; combine the outputs from the first machine learning models to obtain a combined output, input the combined output to a trained second machine learning model, and obtain from the second machine learning model a model prediction about the state of the subject; generate interpretation information indicating relative strengths of contribution to the model prediction from different portions of the measurement data in each channel, the interpretation information for each channel being generated based on a weighted sum of the extracted feature maps output from the respective first machine learning model; and output the generated interpretation information.

Embodiments of the disclosure will now be further described, merely by way of example, with reference to the accompanying drawings.

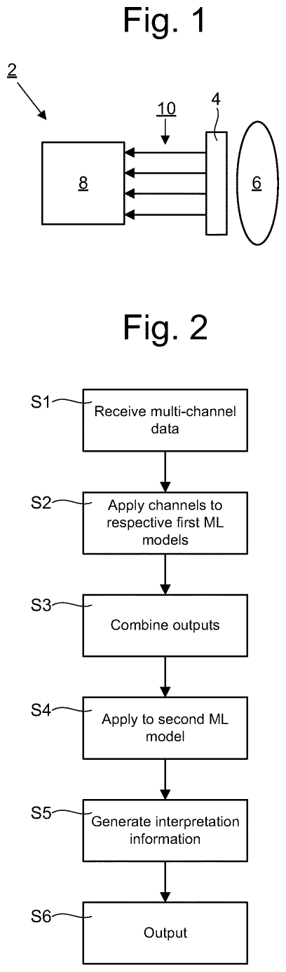

FIG. 1 depicts a measurement system for obtaining multi-channel measurement data from a subject.

FIG. 2 depicts a framework for a method of extracting information from multi-channel measurement data.

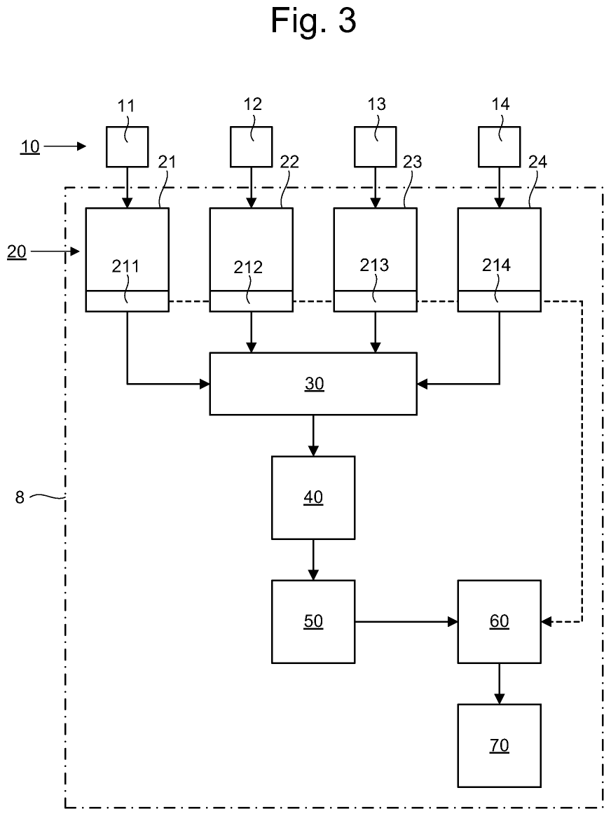

FIG. 3 schematically illustrates the method of FIG. 2.

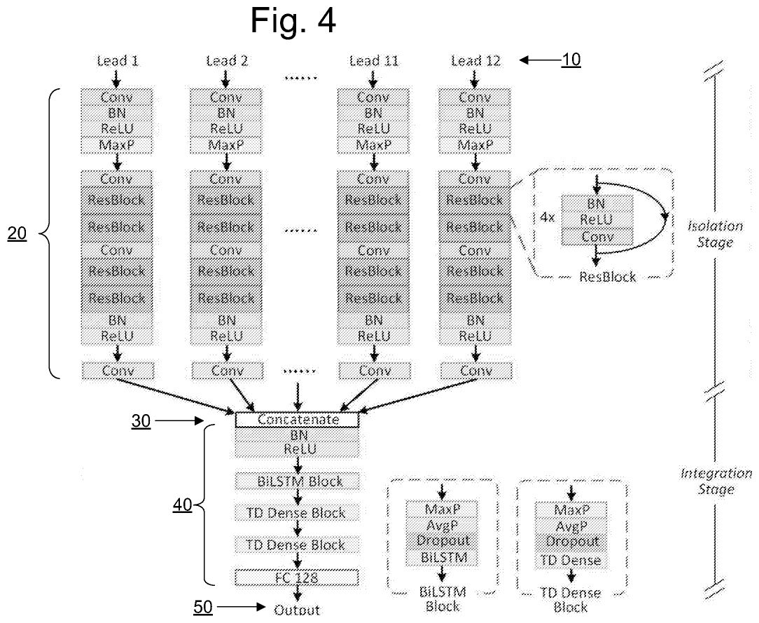

FIG. 4 depicts example detail in upstream portions of the method of FIG. 3, including details of machine learning stages.

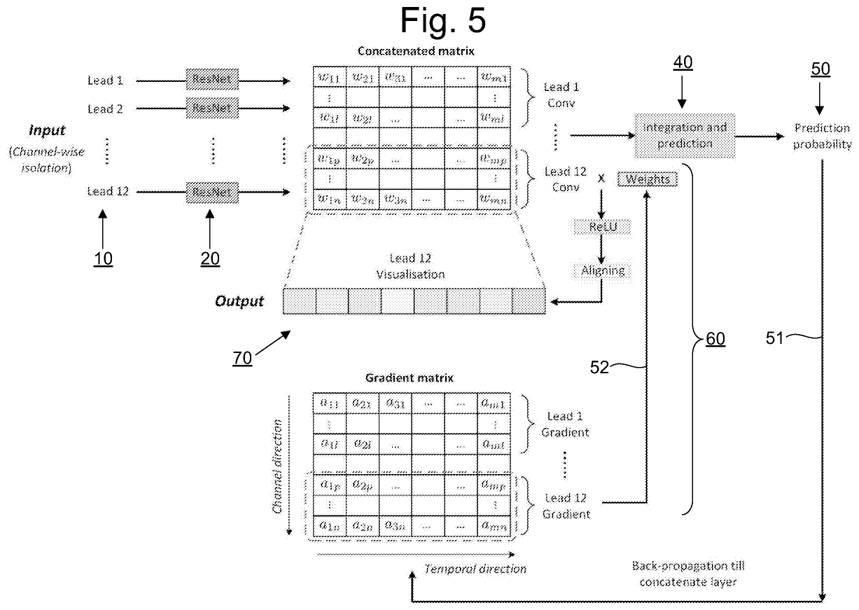

FIG. 5 depicts example detail in downstream portions of the method of FIG. 4, including details of class activation mapping stages.

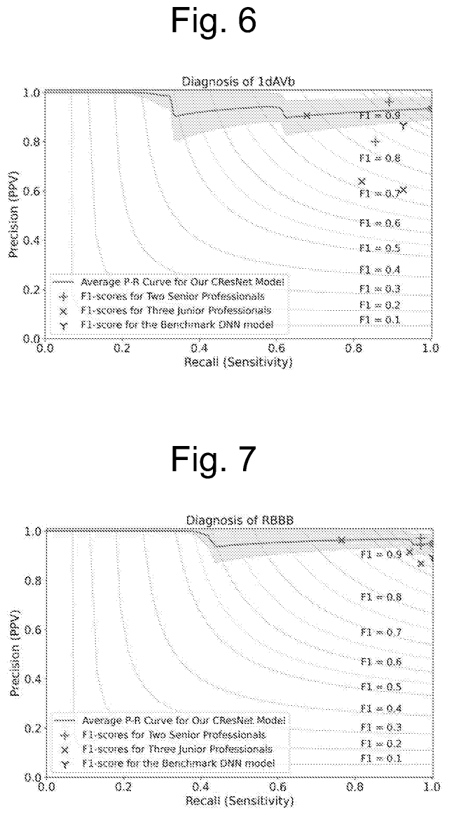

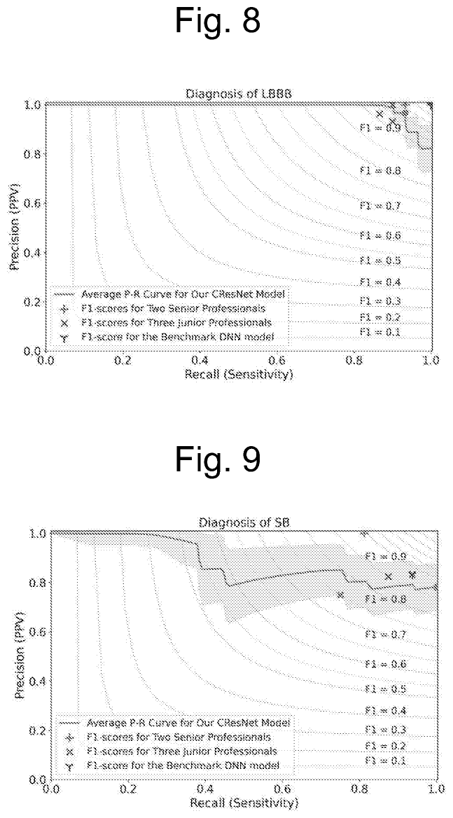

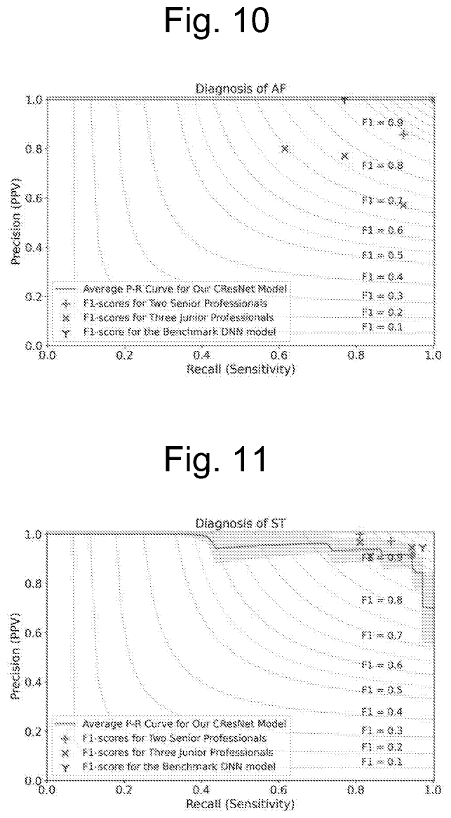

FIGS. 6-11 are graphs depicting performance comparisons for the diagnosis of ECG abnormalities, including (a) 1dAVb (FIG. 6), (b) RBBB (FIG. 7), (c) LBBB (FIG. 8), (d) SB (FIG. 9), (e) AF (FIG. 10), and (f) ST (FIG. 11). These figures show the precision-recall (P-R) curves for the performance of the method shown in FIG. 3, referred to herein as the CResNet model, evaluation results from five cardiology professionals, and the result of a benchmark DNN model (Antônio H Ribeiro et al. “Automatic diagnosis of the 12-lead ECG using a deep neural network”. Nature Communications 11.1 (2020), pp. 1-9). The solid lines are the average P-R curves for the diagnosis of arrhythmias, and the shading areas represent standard deviations obtained by the bootstrap method. The circular dots correspond to the F1-scores for the CResNet model, the ‘+’ symbols are used to denote F1-scores for the two senior professionals, the ‘X’ for the three junior professionals, and the ‘Y’ for the benchmark DNN model. The contour plots show the iso-F1 curves with a constant value for each curve, and a point closes to the ideal score of ‘1’ in the top-right corner indicating a higher F1-score.

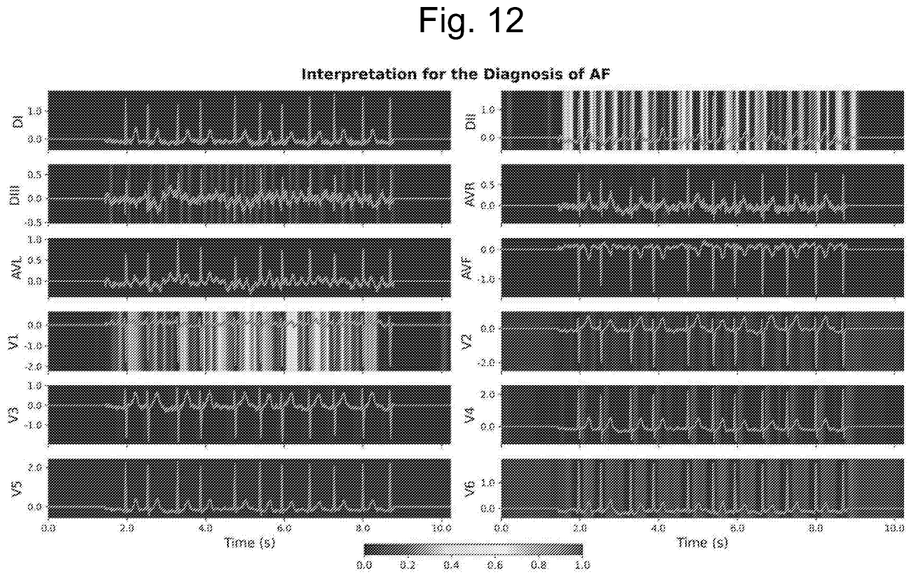

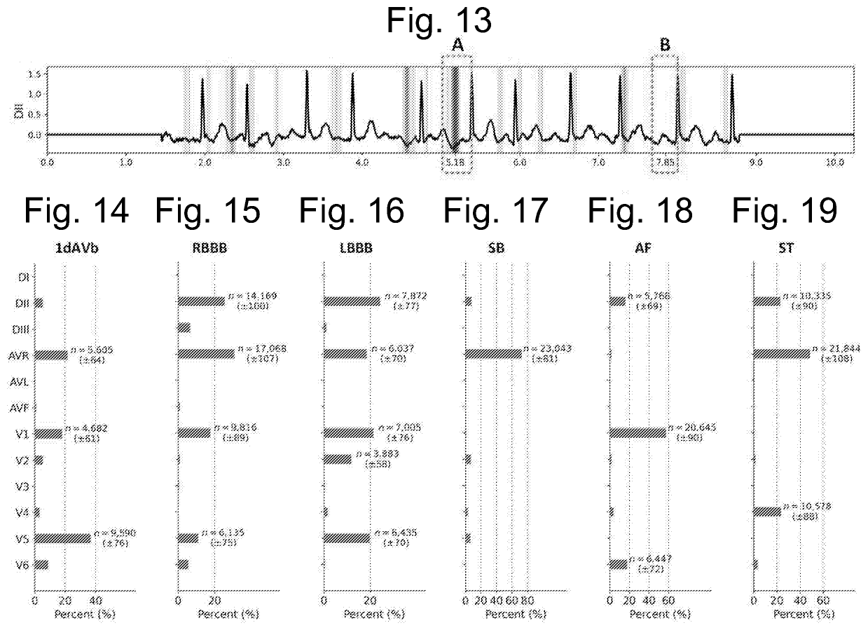

FIGS. 12-19 are graphs illustrating use of the method of FIGS. 4 and 5 for extracting information from multi-channel ECG measurement data, allowing in particular interpretation using the method of FIG. 5 of a diagnosis obtained using the method of FIG. 4, in this case of atrial fibrillation (AF). FIG. 12 depicts calculated heatmaps for the diagnosis of AF using 12 ECG leads, with the shading scale ranging from left to right indicating the increasing weights of data importance. FIG. 13 shows the heatmap of the DII lead in FIG. 12 refined by removing background heatmap shading corresponding to values less than 0.4. Segments A and B show the inconsistent morphologies in the locations of P waves in the DII lead. FIGS. 14-19 show distributions of dominant ECG leads for the diagnosis of: 1dAVb (FIG. 14), RBBB (FIG. 15), LBBB (FIG. 16), SB (FIG. 17), AF (FIG. 18), and ST (FIG. 19). The number of occurrences when the dominant lead accounts for more than 10% of all the 12 ECG leads are annotated onto the graphs. The number of occurrences is presented as mean and standard deviation calculated by bootstrap method.

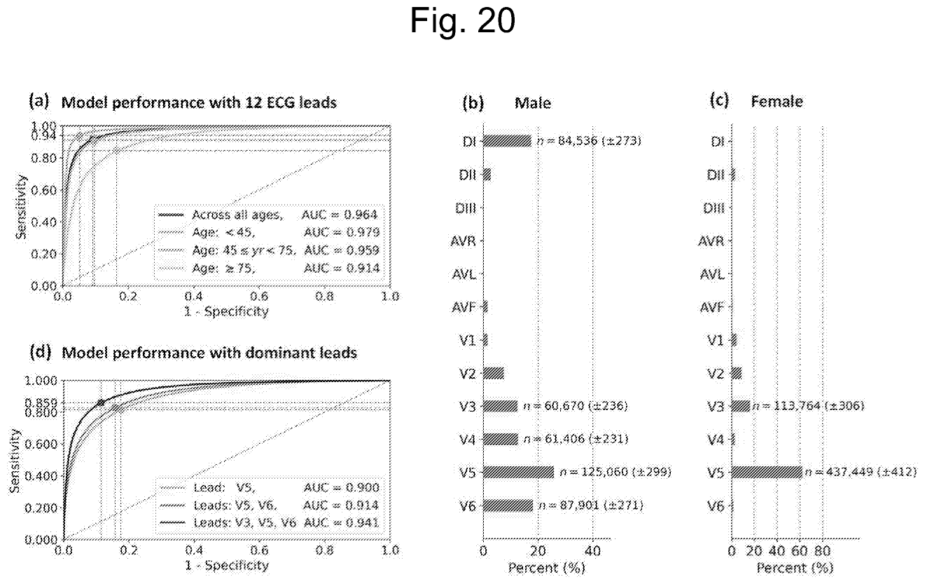

FIG. 20 depicts performance of the CResNet model and lead importance for gender identification using the model. (a) Performance comparison of the CResNet model for gender identification using 12-lead ECG recordings in different age groups. (b) Distributions of dominant leads for identifying male subjects. (c) Distribution of dominant leads for identifying female subjects. (d) Performance comparison between different dominant ECG leads. In each of the receiver operating characteristic (ROC) curves ((a) and (d)), the dot point indicates the optimal cut-off point for the sensitivity and specificity calculated by the G-mean method.

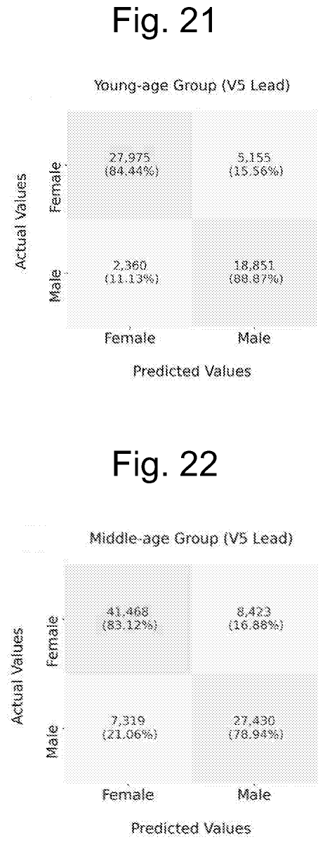

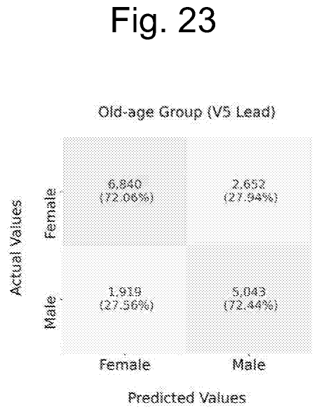

FIGS. 21-23 show confusion matrices for gender identification using the dominant V5 lead in different age groups, including: the young-age group (yr<45) (FIG. 21); the middle-age group (45≤yr<75) (FIG. 22); and the old-age group (yr≥75) (FIG. 23).

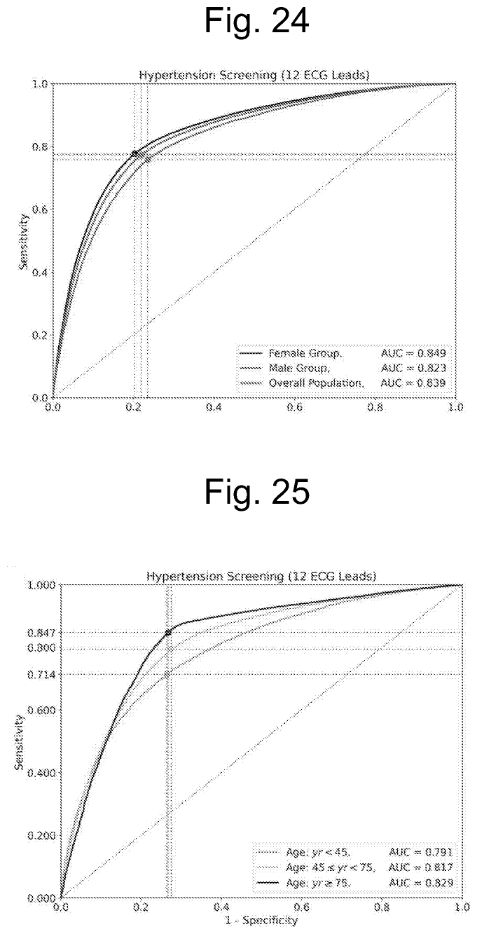

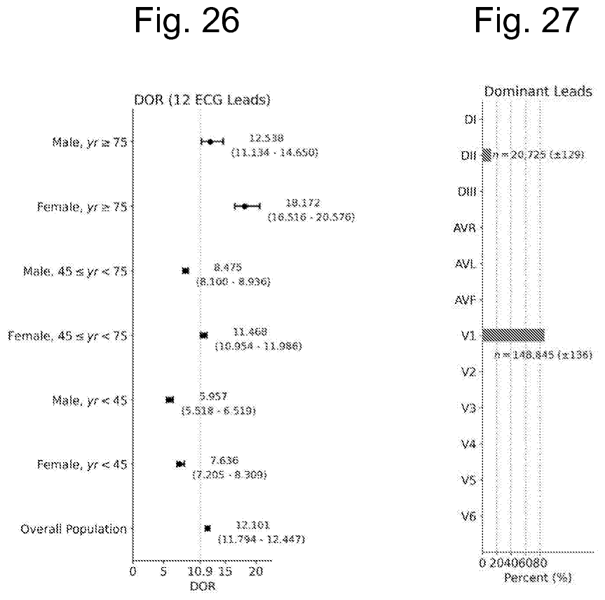

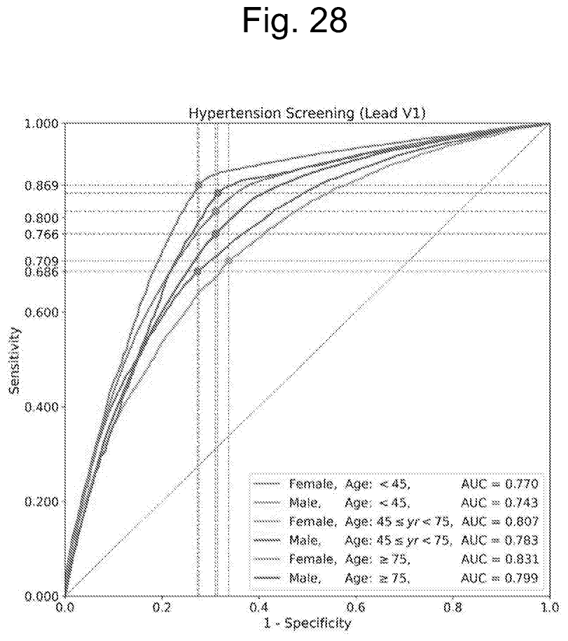

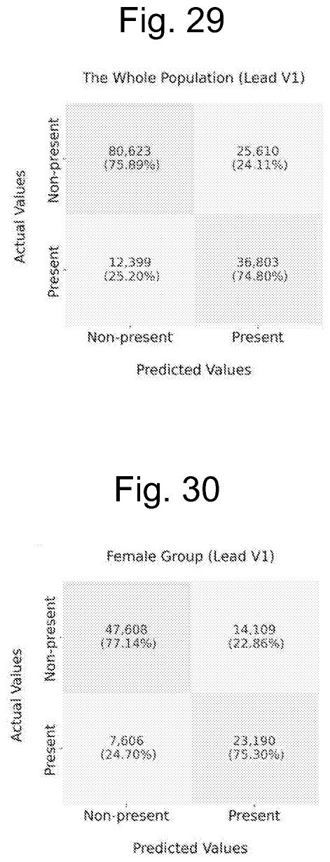

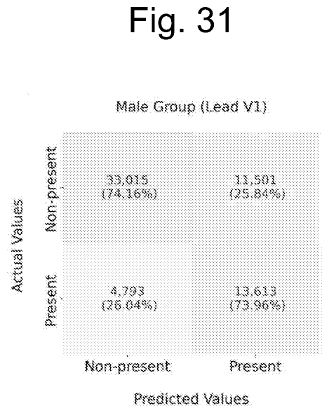

FIGS. 24-31 depict CResNet model performance and lead importance for hypertension screening using the model. FIG. 24 is a performance comparison of the CResNet model for hypertension screening using 12-lead ECGs in terms of gender differences. FIG. 25 is a performance comparison in terms of age differences using 12-lead ECGs. FIG. 26 depicts diagnostic odds ratios (DOR) with 95% CI for hypertension screening in different populations. FIG. 27 depicts distributions of the dominant ECG leads (mean±standard deviation). FIG. 28 is a performance comparison of hypertension screening using the dominant V1 lead. FIGS. 29-31 are confusion matrices for hypertension screening using the dominant V1 lead in different population groups, including: the whole population (FIG. 29); the female group (FIG. 30); and the male group (FIG. 31). The confidence interval and standard deviation are calculated by bootstrap method.

Methods of the present disclosure are computer-implemented. Each step of the disclosed methods may therefore be performed by a computer. The computer may comprise various combinations of computer hardware, including for example CPUs, RAM, SSDs, motherboards, network connections, firmware, software, and/or other elements known in the art that allow the computer hardware to perform the required computing operations. The required computing operations may be defined by one or more computer programs. The one or more computer programs may be provided in the form of media or data carriers, optionally non-transitory media, storing computer readable instructions. When the computer readable instructions are read by the computer, the computer performs the required method steps. The computer may consist of a self-contained unit, such as a general-purpose desktop computer, laptop, tablet, mobile telephone, smart device (e.g., smart TV), etc. Alternatively, the computer may consist of a distributed computing system having plural different computers connected to each other via a network such as the internet or an intranet.

FIG. 1 schematically depicts a measurement system 2 according to an example of the disclosure. The measurement system 2 comprises a measurement apparatus 4. The measurement apparatus 4 comprises elements capable of performing measurements on a subject 6 to obtain measurement data 10. The subject 6 may be a human or animal. The measurements are multi-channel measurements and provide corresponding multi-channel measurement data 10. The multi-channel measurement data 10 may comprise data derived from simultaneous performance of measurements having different measurement types, with each measurement type corresponding to a different respective one of the channels. The different measurement types may for example comprise measurements obtained from multiple electrocardiogram (ECG) channels (e.g., multiple ECG probes or leads). Each ECG channel corresponds to a different respective one of the measurement types. Alternatively or additionally, the different measurement types may comprise measurements obtained from multiple electroencephalogram (EEG) channels (e.g., multiple EEG probes or leads). Each EEG channel corresponds to a different respective one of the measurement types in this case. Alternatively or additionally, the different measurement types may comprise measurements obtained from multiple photoplethysmography (PPG) channels (e.g., multiple PPG probes or leads). Each PPG channel corresponds to a different respective one of the measurement types in this case.

The measurement system 2 comprises a data processing unit 8 comprising a processor configured to perform methods of the disclosure. These methods extract information from the multi-channel measurement data 10 and are described in further detail below.

An example framework for the method performed by the measurement system 2 is shown in FIG. 2, with more detailed example implementation details shown in FIGS. 3-5. The example architectures described involve channel-wise deep residual networks and may be referred to below as CResNet.

In step S1, multi-channel measurement data 10 is received from measurements performed on a subject 6. The multi-channel measurement data 10 may take any of the forms discussed above or other forms. The multi-channel measurement data 10 may comprise time series data. In FIG. 3, the measurement data 10 comprises data units 11-14 from four different channels. Each of the data units 11-14 may comprise time series data from a different measurement probe or lead, such as from a different ECG lead.

In step S2, the measurement data 10 is provided as input to a set 20 of trained first machine learning models 21-24. In the example of FIG. 3, the set comprises four first machine learning models 21-24. Each channel of the measurement data 10 is provided exclusively to a different respective one of the first machine learning models 21-24. In the example of FIG. 3, data unit 11 from the first channel is provided to first machine learning model 21, data unit 12 from the second channel is provided to first machine learning model 22, data unit 13 from the third channel is provided to first machine learning model 23, and data unit 14 from the fourth channel is provided to first machine learning model 24. Each channel is thus isolated from every other channel at this stage. Each first machine learning model only receives and processes data from its own respective channel.

The first machine learning models 21-24 output respective sets of extracted feature maps. One set of extracted feature maps is output for each of the channels. Each set of extracted feature maps comprises one extracted feature map for each of a plurality of convolution kernels. As depicted schematically in FIG. 3, the outputs from the first machine learning models 21-24 may be provided from respective convolution layers 211-214 of the first machine learning models 21-24.

Convolution kernels are well-known features of machine learning models. They are applied in a machine learning “convolution” operation to input data, such as image data, to extract features from the input data. Convolution kernels may be provided in the form of matrices that operate on the input data. A convolution matrix may be smaller than the input data and be applied multiple times in a scanning operation over the input data to provide an extracted feature map formed from the values obtained. Different convolution kernels will be trained to extract different features. Convolution kernels may also be referred to as convolution matrices, convolution masks, or convolution filters. The operation of a convolution kernel may be viewed as a filtering operation that down-weights or removes portions of input data that do not appear to correspond to the feature of interest. For example, in a machine learning model trained to detect faces in an image, a convolution layer might use kernels to extract aspects of images relevant to deducing that a face is present, such as a kernel that filters out all elements except features that look like cars (to provide a feature map showing locations of features that look like cars), a kernel that filters out all elements except features that look like eyes, etc.

The first machine learning models 21-24 may be deep learning models. Residual neural networks have been found to work particularly well. FIG. 4 depicts an example implementation found to work particularly well and presented in the context of analysing ECG data. The same architecture could however be applied to other multi-channel measurement data.

In the example of FIG. 4, the measurement data 10 has 12 channels, with each channel corresponding to one ECG lead. The measurement data 10 from each channel is input to a separate one of a set 20 of first machine learning models as described above. The measurement data 10 from each channel is input to a convolution (Conv) layer of the respective first machine learning model of the set 20. The Conv layer has 16 kernels configured to learn latent features from the measurement data, which in this example is raw data from a respective one of the ECG leads. Each Conv layer is followed by a batch normalisation (BN) layer, a rectified linear unit (ReLU) activation layer, and a max pooling layer. Next, the first machine learning models use four residual blocks to learn deep features from each lead. Each of the residual blocks comprises four repeated modules with the BN, ReLU, and Conv layers. In the first two residual blocks, the Conv layer has 16 kernels with a width size of 16. In the remaining two residual blocks, the Conv layer has 48 kernels and a width size of 48. After the second residual block, a Conv layer with 48 kernels is used to align dimensions with the following third residual block. At the end of each channel, a Conv layer with 48 kernels is used to finish feature learning for the ECG lead. These final Conv layers corresponds to the Conv layers 211-214 depicted in FIG. 3.

In step S3, outputs from the set 20 of first machine learning models 21-24 are combined. As explained above, the outputs comprise extracted feature maps for each of the channels, so combining the outputs from the set provides a larger set of extracted feature maps. In one class of arrangement the sets of extracted feature maps corresponding to the outputs from the set 20 of first machine learning models 21-24 are represented as matrices and the combined output comprises a concatenated feature matrix 30 representing the sets of extracted feature maps.

In step S4, the combined outputs from step S3 are inputted to a trained second machine learning model 40. The second machine learning model 40 uses the combined outputs to provide a model prediction about the state of the subject 6. Unlike the set 20 of first machine learning models 21-24, the second machine learning model uses information from all of the channels. The second machine learning model 40 may thus be trained to learn relationships between different channels of the measurement data 10. The second machine learning model 40 may also be trained to learn temporal information. The second machine learning model 40 may comprise a recurrent neural network for example. The method has been found to work particularly well using a long short-term memory (LSTM) approach, preferably a bi-directional long short-term memory (BiLSTM) approach.

In the example of FIG. 4, the processing performed in steps S1 and S2 is referred to as an “Isolation Stage”. This is because this stage of the processing involves learning features from the isolated input channels. By contrast, the processing of steps S3 and S4 is referred to as an “Integration Stage” because this stage of processing involves stepwise integration of the features to learn elaborate relationships between different ECG leads. In the example of FIG. 4, a concatenated feature matrix is generated by concatenating the learned features from each of the isolated channels as described above (i.e., by combining the extracted feature maps from each of the first machine learning models/channels). As there is only one pooling layer used for each input channel in the isolation stage, the temporal dimension of the generated concatenated feature matrix in the concatenate layer is half the size (along the time dimension) of the input ECG recording.

The last Conv layer in each channel has the size of 48 kernels, and the generated feature matrix has a dimension of 576, which is obtained by concatenating Conv layers in the 12 ECG leads. Thus, the concatenated feature matrix represents 576 distinct extracted feature maps (one for each kernel for each channel).

In the example of FIG. 4, relationships between different ECG leads are learned using a bidirectional long short-term memory (BiLSTM) block and two time-distributed dense layer (TD Dense) blocks. Both the BiLSTM block and TD Dense blocks comprise a max pooling layer (MaxP), an average pooling layer (AvgP), and a dropout layer. The BiLSTM block comprises two LSTM layers, one having a forward direction and the other having a reverse direction. Each LSTM layer has 64 cells in the hidden state. For the two TD Dense blocks, the first block has 64 units and the second block has 32 units. Layers of the TD Dense block are then flattened and followed by a fully connected layer with 128 units. Finally, a sigmoid function is used to calculate probability for the output of model prediction.

In step S5, interpretation information is generated based on the outputs from the first machine learning models 21-24. The interpretation information indicates relative strengths of contribution to the model prediction from different portions of the measurement data in each channel. The interpretation information for each channel is generated based on a weighted sum of the extracted feature maps output from the respective first machine learning model. The weighted sum for each channel may be referred to as a weighted activation map or heatmap. Class activation mapping is a well-known technique in machine learning at a general level for producing weighted activation maps. It is commonly used in the context of image analysis to indicate which regions of an image a trained machining learning model is “looking at” when making a prediction of a given class. Thus, if the trained machine learning model were trained to detect faces, the weighted activation map may contain pixels with relatively high weights in regions of the image that contain features highly correlated with an image containing a face (such as noses, eyes, etc.). In the context of examples of the present disclosure, the weighted activation map may indicate which portions of the measurement data for a given channel contribute most to a model prediction of a particular class. For example, if the model prediction is that a particular ECG abnormality is present, the weighted activation map for a particular channel of the measurement data may indicate which time portions of time series data of that channel contain information most relevant to predicting the presence of the ECG abnormality in question. The class activation mapping thus provides insight into how the machine learning process makes the conclusions that it does. This can enhance clinical understanding and/or improve acceptance of model predictions provided by the machine learning process.

Generating the interpretation information using a weighted sum of the outputs from the first machine learning models 21-24 rather than from the second machine learning model provides information about which portions of the measurement data are most relevant on a channel-by-channel basis even though the machine learning methodology as a whole takes account of relationships and interactions between different channels of the measurement data (via the second machine learning model). An advantageous balance is found that achieves both high quality model predictions (by taking account of relationships between channels) and high quality feedback about which portions of the measurement data are relevant to the model predictions (via the channel by channel calculation of weighted sums).

The framework used to generate the interpretation information may be referred to as an interpretation model. In one class of implementation that has been found to work particularly well, the interpretation model is implemented using a refined gradient-weighted class activation mapping (Grad-CAM) module. The Grad-CAM assumes that the last convolutional (Conv) layer in a deep learning model represents higher-level visual content of the input data. Then, the model calculates the gradient information with respect to the last Conv layer, and uses it to represent the importance of each kernel for the decision making.

Formally, for the input data X and corresponding label y∈ [symbol] , a deep learning model with convolutional neural networks builds mapping for the input data and output label, f:X→y. The Grad-CAM model first computes the gradient score for class y^{c} with respect to the feature map W in the last Conv layer,

$${α}_{k}^{c} = {\overbrace{\frac{1}{Z} {\sum }_{i} {\sum }_{j}}}^{Global average pooling} {\underbrace{\frac{\partial {y}^{c}}{\partial {w}_{i , j}^{k}}}}_{Backprop gradients} ,$$

where,

$${ω}_{i , j}^{k}$$

is the element of feature map W in the last Conv layer,

$${α}_{k}^{c}$$

is the calculated weight, which is used to weight the importance of the k^{th} kernel in the feature map.

Next, a coarse localisation heatmap can be obtained by a weighted combination of feature maps, and it is followed by an activation function,

$${L}_{Grad - CAM}^{c} = Re LU {\underbrace{( {\sum }_{k} {α}_{k}^{c} {W}^{k} )}}_{Linear combination} .$$

where, ReLu(⋅) is the rectified linear unit function, which is used to find a positive influence on the class of interests; matrix

$${L}_{Grad - CAM}^{c}$$

is the calculated heatmap for the y^{c} class, and the calculated heatmap has the same dimension of the kernel size.

FIG. 5 depicts an example implementation of the class activation mapping based on the Grad-CAM approach discussed above. This implementation is an example of a class of embodiment that uses back propagation to compute a gradient of the model prediction with respect to the combined outputs from the first machine learning models. In FIG. 5 the back propagation is indicated by arrow 51 and an example of the gradient matrix is depicted. In this example, the computation of the gradient of the model prediction with respect to the combined outputs provides a gradient matrix having dimensions corresponding to (e.g., the same as) the concatenated feature matrix. The gradient matrix may thus have the same dimensions as the concatenated feature matrix with each element in the matrix representing a gradient of the model prediction with respect to the corresponding element in the concatenated feature matrix. Thus, the magnitude of each element in the gradient matrix provides a measure of how influential that element was in making the model prediction. If the elements in layers of the gradient matrix corresponding to a particular kernel (i.e., the extracted feature maps obtained by applying that kernel) have on average high values then one can conclude that that kernel is an important one for determining the model prediction. The calculation of weightings for kernels may thus comprise, for each kernel, averaging elements of the gradient matrix corresponding to the kernel.

In arrangements of this type, as exemplified in FIG. 5, the method may further comprise using the computed gradient (i.e., the gradient matrix) to calculate weightings (which may also be referred to as weights) (arrow 52) for kernels used to provide extracted feature maps in the combined outputs. Each weighting corresponds to one of the kernels and quantifies a relative strength of contribution to the model prediction of extracted feature maps provided by that kernel. Thus, as described above, if the gradient matrix indicates that a particular kernel extracts features that contribute strongly to the model prediction that kernel will be assigned a higher weighting than kernels that extract less relevant features. The weightings may then be used to generate the weighted activation map for each channel of the measurement data (i.e., the weightings are used as the basis for the overall weightings of the weighted activation map).

In some arrangements, each weighted activation map is generated by calculating a sum of the extracted feature maps corresponding to the channel of measurement data of the weighted activation map. The extracted feature maps in the sum are weighted according to the weightings calculated for the respective kernels (e.g., with higher weightings being given to the more influential kernels). In the example shown in FIG. 5, the kernels are weighted using the averaged gradient scores, which are then filtered by a ReLu function.

In step S6, the interpretation information indicating relative strengths of contribution to the model prediction from different portions of the measurement data is output (e.g., as a data stream, as data to be stored, as visual information on display, etc.). To make the interpretation information easily accessible, the generating and outputting of the interpretation information may comprise dimensionally aligning the weighted activation maps to the measurement data. In the example of FIG. 5, this is done after the filtering by the ReLu function. Thus, if the weighted activation maps are smaller than the measurement data along one dimension, for example along the time axis for time series data, the weighted activation maps may be stretched out until they have the same size as the measurement data and can thus be overlaid on the measurement data. This creates an easily accessible visualisation of the measurement data showing clearly which parts of the measurement data are most relevant for prediction of a given class (e.g., most relevant to predicting a particular ECG anomaly). For high resolution in the dimensionally aligned weighted activation maps it is desirable for the dimensional alignment to be as small as possible. This can be achieved by adapting the first machine learning models. In preferred examples, the first machine learning models 21-24 are configured such that at least one dimension of the extracted features maps is at least 0.2 times, optionally at least 0.3 times, optionally at least 0.4 times, optionally at least 0.5 times, a corresponding dimension of the measurement data. As mentioned above, the dimensional alignment may be performed along a time dimension for time series measurement data, optionally exclusively along the time dimension. Additionally or alternatively, the dimensional alignment may be performed along one or more other dimensions of the measurement data.

The magnitude of the required dimensional alignment may be kept low by limiting the number of pooling layers in each of the first machine learning models 21-24. Preferably, each first machine learning model 21-24 has less than or equal to two pooling layers. In the example discussed above with reference to FIG. 4 each first machine learning model has only a single pooling layer, which in that example resulted in the concatenated feature matrix being half the size of the input measurement data along the time axis.

An output of the model prediction may also be provided in step S6. The model prediction may be single dimensional (e.g., a probability of a particular condition or diagnosis, such as hypertension) or multi-dimensional (e.g., a vector comprising probabilities of multiple different conditions or diagnoses, such as probabilities of different types of ECG abnormality). As will be demonstrated in the examples given below, the approach has been found to be particularly effective for obtaining information about hypertension and ECG abnormality.

### Further Example Details and Demonstrations of Performance

In the examples discussed below, the CResNet architecture of FIGS. 4 and 5 is applied to three tasks, i.e., ECG abnormality diagnosis, gender identification, and hypertension screening. The CResNet model was trained independently for each of the three tasks, whilst keep the model architecture and hyperparameters the same for all the three tasks, i.e., the number of neurons, activation function, optimizer, batch size, and epochs. For the first task, the CResNet model has an output vector (the model prediction) consisting of six values providing respective estimations concerning the presence of six types of ECG abnormality. For the second task, the CResNet model has an output of a single value, indicating the probability of male or female. For the third task, the CResNet model also has an output of a single value, indicating the probability of hypertension presented for the subject.

The neural network was trained using the loss of binary cross-entropy, which was minimized by the Adam optimizer with default parameters. Hyper-parameters of the network architecture were chosen via a combination of grid search and manual tuning with the following considerations, the number of residual blocks {2, 4, 8}, kernel size for the Conv layer {16, 32, 48, 64}, the number of BiLSTM blocks {1, 2, 4}, the size of pooling layers {2, 4}, dropout rate of {0, 0.2, 0.5, 0.6}, the mini batch size of {32, 64, 128}, initial learning rate of {10⁻², 10⁻³, 10⁻⁴}, the number of epochs without improvement in plateaus between 7 and 15, which would result in a reduction of the learning rate by a factor of 10. After tuning the parameters with 300K samples a small scale of the dataset, we set a learning rate of 10⁻⁴ and use the whole dataset to train the model with a mini batch size of 128 samples, and the maximum number of epochs was set as 70. During the model training, a holdout set with 10% of the data was used for the validation. We tried different configurations of the model development, especially in the feature integration stage, such as the BiLSTM, LSTM, and TD Dense layers; and found that the combination of BiLSTM with two TD Dense layers shows good performance for the diagnosis. To reduce the effect of imbalanced classes in the dataset, we weighted each sample by multiplying a score of 2*log(n_{ECG}_{_{S}}/n_{Class}), where n_{ECG}_{_{S}} indicates the total number of samples, and n_{Class} is the size of samples in the class. A total of 20 Nvidia V100 GPUs in a high performance computing platform were used to train the CResNet model, which is located at the Institute of Biomedical Engineering, Department of Engineering Science, University of Oxford.

### Data Acquisition and Annotation

The present study uses a dataset consisting of standard 12-lead ECG recordings collected by the Telehealth Network of Minas Gerais (TNMG), a public healthcare system to provide tele-consultation and tele-diagnosis for 811 municipalities in the state of Minas Gerais, Brazil. The ECG recordings were mostly collected in primary care facilities during clinic visits between 2010 and 2016, which were performed either using the tele-electrocardiogram machine of model TEB (Tecnologia Eletronica Brasileira, Sao Paulo, Brazil), or the ErgoPC 13 (Micromed Biotecnologia, Brasilia, Brazil).

The ECG tests were recorded for a duration of 7 to 10 seconds with sampling frequencies ranging from 300 to 600 Hz. To ensure consistency of the data format, the recordings were resampled with 400 Hz, and then zero-padded to the length of 4,096 data points. The rescaled ECG recordings were stored in a structured database, namely the Clinical Outcomes in Digital Electrocardiology (CODE). A cohort of 2,322,513 ECG recordings were retrieved from the CODE dataset. We excluded low-quality ECGs (NECGS=6,731) that had zero values for more than 80% of the data points, and used a total of 2,315,782 ECG recordings for the current study.

We obtained electronic health records for subjects in the CODE dataset by performing a link matching between the ECG tests and the national mortality information system, using a standard probabilistic linkage method (FRIL: Fine-grained record integration and linkage software, v.2.1.5, Atlanta, GA). Hypertension in the health records was defined as a systolic blood pressure ≥140 mm Hg, or diastolic blood pressure ≥90 mm Hg, or self-declared use of anti-hypertensive medication. The data were anonymised after the linkage matching.

Annotation of ECG recordings in the CODE dataset was performed by both trained professionals and computerised software using the following procedures, (i) the sampled ECG recordings were first sent by internet to central servers, and a team of trained professionals used standardised criteria to generate free-text ECG reports, which were digitally recognised by a hierarchical free-text machine learning method. The ECG reports were periodically audited by professionals to recognise medical errors and discordant interpretations; (ii) The Glasgow 12-lead ECG analysis program was used to analyse the ECG recordings, and generate the diagnosis results of the Glasgow Diagnostic Statements and Minnesota Code; (iii) The presence of a specific ECG abnormality was automatically considered when there was an agreement between the cardiologist report and the computerised diagnosis result. A manual review was performed when the two sources of diagnosis disagreed.

The holdout testing dataset for model evaluation was independently and rigorously reviewed by two certified cardiologists, and the data label was obtained when annotations from the two professionals were matched. Where annotations did not match, a specialist was introduced to decide the diagnosis. Evaluation results of the two senior professionals are presented in Table 1A. The Cohen's kappa coefficient of the evaluation results from the two senior professionals were also calculated; values are 0.741 for IdAVb, 0.955 for RBBB, 0.964 for LBBB, 0.844 for SB, 0.831 for AF, and 0.902 for ST. These values demonstrate the inter-rater agreement for the two professionals, and these evaluation results were therefore used as the data labels. The testing dataset was also reviewed by three groups of junior cardiology professionals, i.e., two 4^{th} year cardiology residents, two 3^{rd} year emergency residents, and two 5^{th} year medical students. To reduce the bias of ECG evaluation, the two professionals in each of the three groups were asked to annotate half of the testing dataset, and the concatenated performance scores were obtained for the three groups.

TABLE 1A
Performance comparison for the diagnosis of abnormalities using standard 12-lead ECG recordings.

| | F1-score (95% CI) | | | | | | |
|---|---|---|---|---|---|---|---|
| | Junior Professionals | | | Senior Professionals | | DNN models | |
| | Cardio. Rd. | Emerg. Rd. | Medical Sd. | Cardio. #1 | Cardio. #2 | DNN [symbol] Comp [37] | CResNet |
| [symbol] AVb | 0.776 | 0.719 | 0.732 | 0.828 | 0.926 | 0.897 | 0.966 |
| | (0.625-0.889) | (0.578-0.831) | (0.605-0.836) | (0.704-0.925) | (0.844-1.000) | (0.793-0.969) | (0.912-1.000) |
| RBBB | 0.917 | 0.852 | 0.928 | 0.957 | 0.971 | 0.944 | 0.971 |
| | (0.842-0.974) | (0.746-0.939) | (0.852-0.985) | (0.899-1.000) | (0.921-1.000) | (0.881-0.989) | (0.928-1.000) |
| LBBB | 0.947 | 0.912 | 0.915 | 0.966 | 1.000 | 1.000 | 0.949 |
| | (0.875-1.000) | (0.828-0.980) | (0.830-0.983) | (0.907-1.000) | (1.000-1.000) | (1.000-1.000) | (0.882-1.000) |
| SB | 0.882 | 0.848 | 0.750 | 0.897 | 0.897 | 0.882 | 0.865 |
| | (0.743-0.976) | (0.692-0.963) | (0.538-0.889) | (0.750-1.000) | (0.741-1.000) | (0.750-0.976) | (0.727-0.973) |
| A [symbol] | 0.769 | 0.696 | 0.706 | 0.870 | 0.889 | 0.870 | 1.000 |
| | (0.545-0.933) | (0.400-0.875) | (0.500-0.865) | (0.667-1.000) | (0.737-1.000) | (0.667-1.000) | (1.000-1.000) |
| ST | 0.882 | 0.946 | 0.873 | 0.896 | 0.930 | 0.960 | 0.933 |
| | (0.789-0.951) | (0.881-0.989) | (0.778-0.949) | (0.813-0.965) | (0.853-0.987) | (0.904-1.000) | (0.862-0.987) |
| Micro avg | 0.876 | 0.846 | 0.833 | 0.908 | 0.945 | 0.938 | 0.948 |
| | (0.830-0.915) | (0.793-0.892) | (0.789-0.876) | (0.871-0.941) | (0.914-0.970) | (0.910-0.961) | (0.921-0.971) |

[symbol]

Cardio. Rd. [symbol] 4^{th} year cardiology residents [symbol] Emerg. Rd. [symbol] 3^{rd} year emergency residents; Medical Sd. [symbol] 5^{th} year medical students; Cardio. #1 [symbol] the 1^{st} cardiologist; Cardio. #2 [symbol] the 2^{nd} cardiologist; DNN [symbol] Comp [symbol] the state-of-the-art benchmark model developed in {37] [symbol] CResNet [symbol] the DNN model developed in this study. The bold-faced scores denote the best performance for the three junior professionals, two senior professionals, and two DNN models. [symbol]
indicates data missing or illegible when filed

### Statistical and Empirical Analysis of Model Performance

To evaluate the performance of the developed CResNet model on the three tasks, we calculated standard matrices of the testing results for each independent task. We computed the area under the receiver operating characteristic curve (AUC-ROC) to report the model performance; we also calculate the F1-score for the first task, as it has an imbalanced testing dataset, and the score is used to compare the performance of our model with the evaluation results from the cardiology professionals and a state-of-the-art model. We calculated the micro average across different classes to report an overall score of the model performance, which computes the total true positives, false negatives, and false positives to obtain a comprehensive metric. The optimal cut-off point for the sensitivity and specificity scores is obtained by maximising the G-mean value, which is a geometric mean of the two scores. We use the diagnostic odds ratio (DOR) to indicate the model's ability of diagnosis, which is calculated as the positive likelihood ratio (sensitivity/(1−specificity)) to the negative likelihood ratio ((1−sensitivity)/specificity). A value of DOR larger than 1 indicates the model having the discriminatory test performance, with the DOR value correlating positively with better diagnosis performance. We use the bootstrap method (repeated sampling for 1,000 times) to compute the 95% confidence interval (CI) and standard deviation for the calculated indices. We use two-sided McNemar's χ² test to evaluate differences between classification results for paired samples, and use Pearson's χ² test to evaluate differences for unpaired samples. We also calculate Cohen's kappa coefficient to test inter-rater/-model agreement. We consider a p-value of less than 0.05 as statistically significant.

### Results

As discussed above, the present disclosure provides an explanatory deep learning model having two major components: a new architecture with channel-wise deep residual networks (CResNet) to implement the medical diagnosis, and an interpretation module to produce salient features that have been used for the decision-making. To validate the diagnostic and interpretation abilities of the developed model, three independent medical tasks were performed using a large dataset consisting of standard 12-lead ECG recordings (n_{ECG}_{_{S}}=2,322,513) collected from unique individuals during clinic visits (n_{Subjects}=1,558,772).

In the first task, 2,315,782 ECG recordings were used to train the CResNet model to diagnose morphological abnormalities, including the first-degree atrioventricular block (1dAVb), right bundle branch block (RBBB), left bundle branch block (LBBB), sinus bradycardia (SB), atrial fibrillation (AF), and sinus tachycardia (ST). The trained CResNet model is then tested on a holdout ECG dataset, which is rigorously annotated by certified cardiologists.

In the second task, the CResNet model was trained for gender identification using ECGs collected from 1,398,907 subjects (female: 59.78%, n_{Female}=836,267), which is tested with holdout ECGs sampled from 155,435 subjects (female: 59.52%, n_{Female}=92,513).

In the third task, the model was trained to screen hypertension for 1,398,907 subjects (hypertension: 31.66%, n_{Hypertension}=442,918), and tested with 155,435 subjects (hypertension: 31.65%, n_{Hypertension}=49,202).

In both the second and third tasks, only one ECG recording was selected for each individual. When a subject has multiple ECG recordings, the earliest record was used.

In the first task, the CResNet model has a micro average AUC score of 0.998 (95% CI, 0.995-0.999) and an F1-score of 0.948 (95% CI, 0.921-0.971) on identifying the ECG morphological abnormalities. The F1-scores are reported in Table 1A and the performance of our CResNet model is compared with evaluation results from three junior professionals with experience in ECGs, two senior cardiologists, and a state-of-the-art study (Antonio H Ribeiro et al. “Automatic diagnosis of the 12-lead ECG using a deep neural network”. Nature Communications 11.1 (2020), pp. 1-9). It can be seen from Table 1A that the highest evaluation score from the three junior professionals is 0.876 (95% CI, 0.830-0.915). The two senior cardiologists have higher performance than the junior professionals, with the highest F1-score of 0.945 (95% CI, 0.914-0.970). The state-of-the-art benchmark has a score of 0.938 (95% CI, 0.910-0.961). In comparison to the evaluation results yielded by the cardiology professionals, the CResNet model has better performance than the three junior professionals and one senior professional in the diagnosis of 1dAVb (p=0.0433). Furthermore, it significantly outperforms the three junior professionals in the diagnosis of AF (p=0.0412), and has comparable performance with that of the senior cardiologists (p=0.2482).

To show a comprehensive comparison of model performance, the evaluation results from the CResNet model, cardiology professionals, and the benchmark are presented in FIGS. 6-11. The highlighted symbol in each of figures indicates the F1-score for each of the evaluation results, and the (circular) point at top-right corner of each figure is the ideal F1-score for the diagnosis. It can be seen from FIGS. 6-11 that the CResNet model has superior or comparable performance with evaluation results from the comparison studies, suggesting effectiveness of the model on the diagnosis of ECG abnormalities.

Among these abnormalities, the diagnosis of AF particularly has important medical implications, because it is a leading cardiac cause of stroke, heart failure, and mortality. However, it is challenging to obtain a definitive diagnosis of AF with ECG recordings, which is also indicated by the evaluation results as presented in Table 1A. It can be seen from the table that among all the five professionals, the highest F1-score for the diagnosis of AF is 0.889 (95% CI, 0.737-1.000); and the benchmark model from the literature also has moderate performance with a score of 0.870 (95% CI, 0.667-1.000). In contrast, our developed CResNet model successfully identifies all AF in the dataset.

Next, an interpretation of how the decisions that have been made by the CResNet model to diagnose ECG abnormalities is performed. FIG. 12 shows a standard 12-lead ECG recording with AF identified in the test. Five cardiology professionals evaluated the test, and only one of the senior cardiologists and the emergency resident successfully diagnosed the AF; while the other senior and two junior professionals failed to diagnose it. Using the CResNet model, the diagnosis of AF with the ECG recording has a prediction probability of 0.961. To interpret the diagnosis that has been identified by the CResNet model, a heatmap (referred to above as a weighted activation map) was calculated for each of the 12 ECG leads. The heatmap highlights the salient information that has been used for decision making. In FIG. 12, the different colours (shades of grey when depicted in greyscale) indicate weights of data points in the ECG recording, e.g., red colour (right of greyscale) for important information with a high weight, and blue (left of greyscale) for less important data with a low weight. It can be seen from FIG. 12 that the CResNet model uses salient information in the DII and V1 leads for the diagnosis of AF, and has the most important features with red colour in the DII lead.

Notably, the hallmark of AF is the absence of P waves in an ECG recording. However, artifacts or fibrillatory waves can mimic P waves and lead to misdiagnosis. FIG. 13 shows the refined view of the DII lead with background colour (corresponding to lower relevance features) removed, which demonstrates the ECG morphology and salient features that have been used for the diagnosis. It can be seen from FIG. 13 that the P wave is absent in some areas of the ECG morphology, e.g., segment A (around 5.18s); and there are also waves clearly presented in some areas, e.g., segment B (around 7.85s). The inconsistent morphologies in the locations of P waves challenge the diagnosis of AF using the ECG recording. Our developed CResNet model is very flexible in the recognition of P waves, and it highlights important information in segment A rather than segment B, which is consistent with the existing diagnostic criteria. As well as identifying the absence of P waves, the CResNet model also recognises S waves as salient features in the DII lead, and other features in the V1 lead. With combining salient information from different leads in the ECG recording, the CResNet model makes a comprehensive decision with the prediction probability of 0.961 for the diagnosis of AF.

Other than the interpretation for the diagnosis of AF, salient features that are used to diagnose other types of ECG abnormalities have also been identified using the approach. These results demonstrate that the interpretation that has been made by the CResNet model matches well with existing knowledge, but also provides new implications with the identified salient features. For example, the CResNet identifies the absence of Q waves, notched R waves, and T wave inversion in the V6 lead for the diagnosis of LBBB. Furthermore, the CResNet highlights the absence of Q waves and T wave inversion in the DII lead even with higher weights. Combining salient features in the 12 ECG leads, the CResNet model diagnoses the LBBB with a probability of 0.948. In another example, the CResNet model identifies the U waves in the AVR lead, and uses them as important information for the diagnosis of SB. This is consistent with previous observations of prominent U waves in the ECG recording. Apart from identifying U waves in the AVR lead, the CResNet also identifies the downslopes of T waves in DII lead as important information, and the model has a probability of 0.932 to diagnose the SB with combining salient information in the ECG recording.

In a further step, because ECG abnormalities have varied morphologies, the statistical results of dominant leads that are derived from the salient information are presented. First, we filter ECG recordings in the whole dataset with prediction probabilities higher than 0.8, which indicates the CResNet model having confident outputs for the diagnosis of abnormalities. Then, the values of the heatmap are summed for each lead, and the dominant lead with the highest value for the ECG recording is identified. To show distributions of the identified dominant leads, their occurrences and the percentages among all the 12 ECG leads are calculated, and the results can be found in FIGS. 14-19. It can be seen from FIGS. 14-19 that the six types of ECG abnormalities have varied distributions of dominant leads. The 1dAVb has AVR, V1, and V5 as dominant leads; both the RBBB and LBBB have dominant DII, AVR, V1, and V5 leads; the SB has a prominent AVR lead; the AF has three dominant leads of DII, V1 and V6; and the ST has DII, AVR and V4 as dominant leads.

Next, the effectiveness of the identified dominant leads on the diagnosis of ECG abnormalities was investigated. As shown in FIGS. 14-19, the AVR and V1 leads are two representative leads for the ECG abnormalities. We therefore use the two leads to train the CResNet model, and test the performance on the holdout dataset. Table 1B shows the results of the diagnosis using the AVR and V1 leads.

TABLE 1B
Performance comparison for the diagnosis of abnormalities using ECG recordings with dominant leads.

| | Dominant AVR and V [symbol] Leads | | | Dominant DII, AVR, and V [symbol] Leads | | |
|---|---|---|---|---|---|---|
| | Precision | AUC | F1-score | Precision | AUG | F1-score |
| | (95% CI) | (95% CI) | (95% CI) | (95% CI) | (95% CI) | (95% CI) |
| [symbol] AVb | 0.870 | 0.991 | 0.784 | 0.952 | 0.992 | 0.816 |
| | (0.714-1.000) | (0.982-0.998) | (0.632-0.898) | (0.842-1.000) | (0.982-0.998) | (0.667-0.927) |
| RBBB | 0.935 | 0.996 | 0.892 | 0.909 | 0.997 | 0.896 |
| | (0.833-1.000) | (0.992-0.999) | (0.800-0.964) | (0.800-1.000) | (0.993-0.999) | (0.807-0.966) |
| LBBB | 0.966 | 0.978 | 0.949 | 1.000 | 0.993 | 0.947 |
| | (0.889-1.000) | (0.931-1.000) | (0.875-1.000) | (1.000-1.000) | (0.982-1.000) | (0.880-1.000) |
| SB | 0.762 | 0.998 | 0.865 | 0.842 | 0.999 | 0.914 |
| | (0.565-0.947) | (0.995-1.000) | (0.722-0.973) | (0.64 [symbol] -1.000) | (0.996-1.000) | (0.786-1.000) |
| A [symbol] | 0.857 | 0.998 | 0.889 | 1.000 | 0.997 | 0.917 |
| | (0.636-1.000) | (0.993-1.000) | (0.727-1.000) | (1.000-1.000) | (0.992-1.000) | (0.750-1.000) |
| ST | 0.868 | 0.997 | 0.880 | 0.878 | 0.998 | 0.923 |
| | (0.750-0.972) | (0.993-0.999) | (0.786-0.956) | (0.757-0.973) | (0.995-1.000) | (0.844-0.976) |
| Micro [symbol] avg | 0.885 | 0.990 | 0.879 | 0.921 | 0.995 | 0.903 |
| | (0.830-0.937) | (0.982-0.995) | (0.834-0.919) | (0.875-0.962) | (0.992-0.997) | (0.868-0.935) |

*The term of ‘Dominant AVR and V [symbol] Leads’ indicates that the model has inputs with only two ECG leads [symbol] e.g. AVR and V [symbol] leads, rather than using 12 ECG leads. [symbol]
indicates data missing or illegible when filed

It can be seen from the table that the CResNet model achieves an AUC score of 0.990 (95% CI, 0.982-0.995) and an F1-score of 0.879 (95% CI, 0.834-0.919) using the two dominant leads, which is comparable to the best performance of the three junior professionals (p=0.505). In addition to the dominant AVR and V1 leads, the DII lead is also representative for all types of ECG abnormalities. With inclusion of the DII lead, the CResNet model achieves an AUC score of 0.995 (95% CI, 0.992-0.997) and an F1-score of 0.903 (95% CI, 0.868-0.935). In particular, using the DII, AVR, and V1 leads, the model has an F1-score of 0.917 (95% CI, 0.750-1.000) for the diagnosis of AF, and 0.923 (95% CI, 0.844-976) for the diagnosis of ST, which is higher than the scores of 0.889 (95% CI, 0.727-1.000) and 0.880 (95% CI, 0.786-0.956) using the AVR and V1 leads.

The CResNet model was additionally validated on an external dataset retrieved from the PhysioNet/CinC Challenge 2017 (Gari D Clifford et al. “AF classification from a short single lead ECG recording: The PhysioNet/computing in cardiology challenge 2017”. 2017 Computing in Cardiology (CinC) (2017), pp. 1-4). The dataset consists of short single-lead ECG recordings that have been annotated with four classes, i.e., normal sinus rhythm, atrial fibrillation, other alternative rhythms, and noise. The CResNet model was trained using ECG recordings (nECGs=8,528) in the training dataset, and the model was tested on the holdout validation dataset. The CResNet model has a micro average F1-score of 0.884 on the validation dataset. In particular, the model has an F1-score of 0.929 on the diagnosis of AF, and a score of 0.921 on detecting noise signals. Given the widespread noises in ECG recordings, the results indicate robustness of our model for the diagnosis of heart rhythm abnormalities

In the second task, as demonstrated in FIG. 20(a), the CResNet model has an AUC score of 0.964 (95% CI, 0.963-0.965) on gender identification for individual subjects in the holdout testing dataset (n_{Subjects}=155,435). Because features presented in ECG recordings may change over time due to normal ageing, the model performance was investigated in different age groups, i.e., young-age (years (yr)<45, n_{Subjects}=54,341), middle-age (45<yr<75, n_{Subjects}=84,640), and old-age (yr>75, n_{Subjects}=16,454). It can be seen from FIG. 20(a) that the CResNet model has an AUC score of 0.979 (95% CI, 0.977-0.980) on identifying genders for the young-age group, which is higher than the AUC score of 0.959 (95% CI, 0.958-0.961) for middle-age group, and 0.914 (95% CI, 0.909-0.918) for old-age group, suggesting the effect of ageing on the gender identification (p<0.01) using standard 12-lead ECG recordings.

To show the interpretation of gender identification, the salient features in ECG recordings for identifying female and male were visualised. It can be seen from this that the model mainly uses salient information from the DII, V1, and V5 leads for identifying the female subject, which has a prediction probability of 0.971. For identifying the male subject, the model uses the DI, V4, V5, and V6 leads and has a probability of 0.981. The post-hoc method as presented in the first task was then used to analyse the distribution of dominant leads for gender identification. FIGS. 20(b) and (c) present distributions of dominant leads for identifying male and female subjects separately. It can be seen from FIGS. 20(b) and (c) that V5 is the mostly used lead for gender identification by the CResNet model, which is a dominant lead for identifying male subjects (n_{Male}=125,060±299) and female subjects (n_{Female}=437,449±412). Other than the V5 lead, the V3 lead also appears as a dominant lead for identifying male subjects (n_{Male}=60,670±236) and female subjects (n_{Female}=113,764±306).

Next, the model performance of gender identification using the identified dominant leads was investigated, and the comparison results are presented in FIG. 20(d). As the V5 lead is dominant for both male and female subjects, we first only use the V5 lead to identify gender, and the CResNet model obtains an AUC score of 0.900 (95% CI, 0.899-0.902). Confusion matrices of gender identification in different age groups using the dominant V5 lead are shown in FIGS. 21-23. It can be seen that the CResNet model has the highest performance in the young-age group (p<0.01), with an accuracy of 84.44% for identifying female subjects and 88.87% for identifying male subjects. Apart from the V5 lead, it is noted that the V6 is identified as a dominant lead for identifying males, but is less important for females, as shown in FIG. 20(c). Therefore, a combination of V6 with V5 was used to implement the gender identification, and the model has a slightly higher AUC score of 0.914 (95% CI, 0.913-0.915) with the two dominant leads (p<0.01). In a further step, the V3 lead was included to generate a new combination of three dominant leads for gender identification, and the AUC score increases from 0.914 (95% CI, 0.913-0.915) using the V5 and V6 leads to 0.941 (95% CI, 0.940-0.943) using the V3, V5, and V6 leads, indicating the importance of the V3 lead for gender identification (p<0.01).

Comprehensive comparisons of model performance using different combinations of dominant ECG leads for gender identification were also performed. It was found that using the DI, V3, and V5 leads, the CResNet model has the highest performance (p<0.01) with an AUC score of 0.970 (95% CI, 0.969-0.972) and a diagnostic odds ratio (DOR) of 145.891 (95% CI, 139.089-156.331) for gender identification in the young-age group. It is noted that all models have lower performance on identifying genders in the old-age group than in the young-age group (p<0.01), with the highest AUC score of 0.885 (95% CI, 0.880-0.890) in the old-age group using the DI, V3, and V5 leads. The comparison results of model performance suggest the effectiveness of our identified dominant ECG leads for gender identification.

In parallel with the previous two tasks, the third task of hypertension screening was implemented using the CResNet model, and the results of model performance are presented in FIGS. 24 and 25. It can be seen from FIG. 24 that the CResNet model achieves an AUC score of 0.839 (95% CI, 0.837-0.841) and a diagnostic odds ratio (DOR) of 12.101 (95% CI, 11.794-12.447) in screening subjects with hypertension in the testing dataset (hypertension: 31.65%, n_{Hypertension}=49,202). Considering the effects of age and gender on the prevalence of hypertension, the model performance of hypertension screening was investigated in different populations. It can be seen from FIG. 24 that the model achieves an AUC score of 0.849 (95% CI, 0.847-0.852) for hypertension screening in the female group, which is slightly higher than the AUC score of 0.823 (95% CI, 0.820-0.827) in the male group (p=0.011). In terms of age differences, FIGS. 25 and 26 show that the model has the highest performance in the old-age female group (p<0.01), with an AUC score of 0.829 (95% CI, 0.822-0.836) and the DOR of 18.172 (95% CI, 16.516-20.576).

To show the interpretation of hypertension screening, a visualisation was made of the salient features in the 12 ECG leads that have been used to make a decision by the CResNet model. It was found that the CResNet model mostly uses the DII, AVL, and V1 leads to screen hypertension, with particular focus on T waves in the DII and V1 leads. Next, the post-hoc analysis was used to identify dominant ECG leads from the salient features, and investigate their performance on hypertension screening.

It can be seen from the distribution in FIG. 27 that the CResNet model identifies the DII and V1 as dominant ECG leads. In particular, the V1 lead accounts for more than 80% of the occurrences among the 12 ECG leads, which is used to screen hypertension for n_{Hypertension}=148,845±136 subjects. Other than the V1 lead, the DII lead is also identified as a dominant lead to screen hypertension for n_{Hypertension}=20,725±129 subjects.

The dominant V1 lead was therefore used to screen hypertension for individual subjects, and it can be seen from FIG. 28 that the model obtains an AUC score of 0.831 (95% CI, 0.823-0.840) in the old-age female group, which is a similar result to the model performance of 0.839 (95% CI, 0.837-0.841) using 12 ECG leads. FIGS. 29-31 show that the CResNet model has an accuracy of 74.80% on screening hypertension in the whole population using the V1 lead, and it has a higher accuracy of 75.30% in the female group than in the male group. In a further step, two ECG leads were used to screen hypertension with inclusion of the additional DII lead, which achieves the highest AUC score of 0.835 (95% CI, 0.827-0.844) in the old-age female group.

1. A computer-implemented method for extracting information from multi-channel measurement data, the method comprising:
receiving multi-channel measurement data derived from measurements performed on a subject;
providing the measurement data as input to a set of trained first machine learning models, each channel of the measurement data being provided exclusively to a different respective one of the first machine learning models, and obtaining as outputs from the first machine learning models respective sets of extracted feature maps, one set for each of the channels and each set comprising one extracted feature map for each of a plurality of convolution kernels;
combining the outputs from the first machine learning models to obtain a combined output, inputting the combined output to a trained second machine learning model, and obtaining from the second machine learning model a model prediction about the state of the subject; and
generating and outputting interpretation information indicating relative strengths of contribution to the model prediction from different portions of the measurement data in each channel, the interpretation information for each channel being generated based on a weighted sum of the extracted feature maps output from the respective first machine learning model.

2. The method of claim 1, wherein the combined output comprises a concatenated feature matrix representing the sets of extracted feature maps.

3. The method of claim 1, wherein the first machine learning models are deep learning models, optionally comprising residual neural networks.

4. The method of claim 1, wherein the second machine learning model is trained to learn relationships between different channels of the measurement data.

5. The method of claim 4, wherein the second machine learning model comprises a recurrent neural network, optionally a long short-term memory, LSTM, optionally a bi-directional long short-term memory, BiLSTM.

6. The method of claim 1, wherein the generating and outputting of the interpretation information comprises dimensionally aligning weighted activation maps to the measurement data, each weighted activation map representing the weighted sum of the extracted feature maps output from a respective one of the first machine learning models.

7. The method of claim 6, wherein the first machine learning models are configured such that at least one dimension of the extracted feature maps is at least 0.2 times a corresponding dimension of the measurement data.

8. The method of claim 6, wherein the dimensional alignment is performed along a time dimension.

9. The method of claim 1, wherein each first machine learning model comprises only one pooling layer or only two pooling layers.

10. The method of claim 1, wherein the generating interpretation information comprises:
using back propagation to compute a gradient of the model prediction with respect to the combined output from the first machine learning models;
using the computed gradient to calculate weightings for kernels used to provide extracted feature maps in the combined output, each weighting corresponding to one of the kernels and quantifying a relative strength of contribution to the model prediction of extracted feature maps provided by that kernel; and
using the weightings to generate the weighted sum of the extracted feature maps for each channel of the measurement data.

11. The method of claim 10, wherein the extracted feature maps in each weighted sum are weighted according to the weightings calculated for the respective kernels.

12. The method of claim 10, wherein:
the combined output comprises a concatenated feature matrix representing the sets of extracted feature maps;
the computation of the gradient of the model prediction with respect to the combined output provides a gradient matrix having dimensions corresponding to the concatenated feature matrix; and
the calculation of weightings for kernels comprises, for each kernel, averaging elements of the gradient matrix corresponding to the kernel.

13. The method of claim 1, wherein the subject is a human or animal.

14. The method of claim 1, wherein the multi-channel measurement data comprises time series data.

15. The method of claim 1, wherein the multi-channel measurement data comprises one or more of the following: electrocardiogram, ECG, data; electroencephalography, EEG, data; or photoplethysmography, PPG, data.

16. The method of claim 1, wherein the multi-channel measurement data comprises data derived from simultaneous performance of measurements having different measurement types, each measurement type corresponding to a different respective one of the channels.

17. The method of claim 16, wherein the different measurement types comprise one or more of the following:
measurements obtained from multiple ECG channels, each ECG channel corresponding to a different respective one of the measurement types;
measurements obtained from multiple EEG channels, each EEG channel corresponding to a different respective one of the measurement types; or
measurements obtained from multiple PPG channels, each PPG channel corresponding to a different respective one of the measurement types.

18. The method of claim 16, wherein:
the different measurement types comprise measurements obtained from multiple ECG channels, each ECG channel corresponding to a different respective one of the measurement types; and
the model prediction comprises information about ECG abnormality.

19. The method of claim 16, wherein:
the different measurement types comprise measurements obtained from multiple ECG channels, each ECG channel corresponding to a different respective one of the measurement types; and
the model prediction comprises information about hypertension.

20. The method of claim 1, comprising outputting the model prediction.

21. A computer program comprising instructions which, when the program is executed by a computer, cause the computer to carry out the method of claim 1.

22. A computer-readable medium or data carrier signal carrying the program of claim 21.

23. A data processing apparatus comprising a processor configured to perform the method of claim 1.

24. A measurement system for obtaining multi-channel measurement data, comprising:
a measurement apparatus configured to perform multi-channel measurements on a subject to obtain multi-channel measurement data; and
a data processing unit comprising a processor configured to perform the following steps:
provide the measurement data as input to a set of trained first machine learning models, each channel of the measurement data being provided exclusively to a different respective one of the first machine learning models, and obtain as outputs from the first machine learning models respective sets of extracted feature maps, one set for each of the channels and each set comprising one extracted feature map for each of a plurality of convolution kernels;
combine the outputs from the first machine learning models to obtain a combined output, input the combined output to a trained second machine learning model, and obtain from the second machine learning model a model prediction about the state of the subject;
generate interpretation information indicating relative strengths of contribution to the model prediction from different portions of the measurement data in each channel, the interpretation information for each channel being generated based on a weighted sum of the extracted feature maps output from the respective first machine learning model; and
output the generated interpretation information.
